(19) United States
(12) Patent Application Publication
(10) Pub. No.: US 2006/0057232 A1
(43) Pub. Date: Mar. 16, 2006

(54) Method of altering age perception

(76) Inventor: Alan R. Hirsch, Riverwoods, IL (US)

Correspondence Address: WHYTE HIRSCHBOECK DUDEK S C, 555 EAST WELLS STREET, SUITE 1900, MILWAUKEE, WI, 53202, US

(21) Appl. No.: 11/200,542

(22) Filed: Nov. 23, 2005

Related U.S. Application Data
(60) Provisional application No. 60/600,320, filed on Aug. 10, 2004.

(51) Int. Cl. A61K 36/752; A61K 31/11

(52) U.S. Cl. 424/736; 514/699

(57) ABSTRACT

Methods and compositions for altering perception of age of an individual are disclosed.

### CROSS-REFERENCE TO RELATED APPLICATION

This application claims the benefit of U.S. Provisional application Ser. No. 60/600,320, filed on Aug. 10, 2004, the teachings and disclosures of which are herein incorporated by reference.

### FIELD OF THE INVENTION

The present invention relates to altering perception of age by the administration of odorants.

### BACKGROUND OF THE INVENTION

Advances in medicine and public health have allowed the ranks of the elderly to swell. In 1870, only 3% of America's population reached the age of 65. (1) Currently, 12.7% of the population has exceeded this, and by 2020, 20% of the population will be over 65 years old. (1, 2) Despite the demographic imperative of the baby boom generation, the idealized age still remains young. (1) The discrepancy between actual and desired age serves to generate an ego dystonic state, forcing the elderly into a condition of denial, as manifest either by age defying activities (i.e., rigorous exercise programs, bungee jumping, sexual promiscuity) or efforts to negate the effects of the vicissitudes of aging (diet programs, longevity vitamins, etc.).

Behavioral characteristics, which define the elderly, include their clothing, need for visual and mobility aids and the constant specter of failing health, which are intertwined with the aging experience. (1) Perception of age is an integration of multi-sensory domains. Visually manifested signs of aging include accentuated posture (3), reduced limb muscle mass (4), increased trunkal obesity (4, 5), and hyperfunctional facial lines. (3, 4, 5, 6, 7, 8) Acoustic indicators of aging include change in prosody, amount of inflection, (9) and a variety of linguistic dimensions. (10)

Unlike the sensory stimuli above, olfactory generated information has not been studied as an indicator of age. Clinical geriatric experience in extended care facilities and hospitals suggests the infirm, often incontinent; elderly are frequently imbued with a concoction of uriniferous, fecal, and otherwise malodorous aromas. Olfactory evidence of aging has not been addressed amongst the otherwise healthy outpatient population.

Because of the emphasis of youth in today's society with associated ageism (11), there is strong incentive for the elderly to not appear aged. Sensory evidence of youth is demonstrated by the absence of the above sensory signs of aging. Mechanisms used to eliminate these signs include use of cosmetics, Botox shots, cosmetic surgery, hair coloring, adopting current fashion in clothing and slang, and actively participating in traditionally youth-oriented behavior (driving sports cars or Harley-Davidson motorcycles).

Ambient odors have been demonstrated to impact a wide variety of behaviors, including gambling (12), weight loss (13) and sexual arousal (14, 15, 16) (as well as primarily cognitively mediated actions including learning behaviors (17) and perception of external space (18) and weight (19, 73).

It would be useful to provide a means of altering perception of age that does not involve a physical alteration of the body.

### SUMMARY OF THE INVENTION

The present invention relates to methods of using odorants to alter a male's perception of the chronological age of a female. Estimation of age is a primarily cognitively mediated behavior. It has been surprisingly discovered that certain odorants function to influence a male's perception of the chronological age of a female person reflected through an estimation of the female person's age.

The present invention is directed to a method for enhancing a male individual's perception of reduced age of a female individual through the delivery of a composition to the male individual comprising an odorant (aroma) or odorant mixture for inhalation. In particular, the method involves delivering an effective amount of an odorant or odorant mixture to a male subject for inhaling for an effective period of time to cause the male subject's perception of a viewed female's age to be lower than the actual chronological age of the female, preferably lower by at least about 10% or more, or by about 4-10 years. In one embodiment, the method involves administering an odorant or odorant mixture with negative hedonics to a male subject. In a preferred embodiment, the method involves the delivery of a composition comprising a grapefruit odorant, preferably a pink grapefruit odorant, as the primary or dominant odor of the composition.

The method can be effectively practiced, for example, to enhance a female's self-confidence and self-esteem related to a male's negative perception of the female's age, i.e., the female's negative feelings of being considered as old by a male individual. The administration of the odorant composition can also be used in treating a female's social phobias and low self-esteem.

In particular, the method can be utilized on a “viewing” male individual (who inhales the odorant/mixture) to affect their perception of the age of a female individual (“viewed” female individual), particularly a female individual who's age is from about 20 to about 80 years, to be less than the actual age of the female resulting in the male's increased positive perception of the viewed female individual.

According to the method, a composition containing a effective amount of a defined odorant or odorant mixture, preferably a composition containing a grapefruit odorant as the dominant odor, and more preferably a pink grapefruit odorant, is administered to a male individual for inhalation to effectively reduce the male's level of anxiety and elevate the male's sense of well-being resulting in a positive view of physical attributes of a female individual such that the male perceives the age of the female individual to be less than their actual age, preferably about 4-10 years less than the female's chronological age, and more preferably at least about 5-7 years less. The altered perception of the viewing male individual can also generate a positive effect resulting in an anxiety reducing or calming effect on the viewed female individual and increase the viewed female individual's feelings of well being associated with a positive perception of their age.

In another embodiment, the composition contains a mixture of pink grapefruit, vanilla, and baby powder odorants.

In another embodiment, the method can be utilized in treating a female individual diagnosed with low self-esteem, a social phobia, and/or mild depression, by administering an effective amount of a composition composed of one or more odorants, including a grapefruit odorant as the dominant or primary odor or essence, to a male individual for inhalation, such that the male individual inhaling the odorant perceives the age of the female individual being treated (“viewed” female individual) to be less than the actual age of the female, resulting, for example, in a positive affect on and reducing the anxiety of the viewed female individual. One preferred odorant composition is composed of one or more hedonically negative odorants. A preferred composition comprises an effective amount of a grapefruit odorant, preferably a pink grapefruit odorant, as the dominant odor within the composition. In a preferred embodiment, the composition contains a mixture of pink grapefruit, vanilla, and baby powder odorants. The method can be combined with behavior modification therapy directed to the female individual.

In another embodiment, the method can be utilized in reducing anxiety and elevating a sense of well-being in a male subject diagnosed with low self-esteem, a social phobia, and/or mild depression. The method includes administering to the male subject for inhalation, an effective concentration of an odorant composition formulated with one or more odorants of which a grapefruit odorant, preferably a pink grapefruit odorant, is the dominant odor of the composition, for an effective time period such that anxiety is reduced and/or a sense of well-being is enhanced in the male subject, and the male subject perceives the chronological age of one or more female subject(s) as being less than their actual age, preferably up to about 10 years less than the actual age, and preferably at least about 4-7 years less.

The method of the invention can also be used as an adjuvant or aid to assist in treatments for age perception problems including social phobia, low self-esteem, and/or mild depression by reducing anxiety associated with feelings of being old, and/or by enhancing a positive perception of age.

The invention further provides a composition containing an effective amount of a hedonically negative odorant as the dominant odor, preferably a grapefruit odorant, and most preferably a pink grapefruit odorant, to effectively alter the perception of the male subject of the chronological age of a female individual viewed by the male subject within about 1-5 minutes following inhalation of the composition, whereby the age of the female individual is perceived by the male subject to be up to about 10 years less than the actual or chronological age, preferably at least about 4-7 years less, and preferably at least about 6 years less. In a preferred embodiment, the composition contains a mixture of pink grapefruit, vanilla, and baby powder odorants. The composition can be formulated as a spray, a cream, a lotion, and the like, and can be contained within a liquid pump device, aerosol spray device, lidded container, towlette sealed within a foil pouch, or other suitable vessel such as those known and used in the art. The composition is preferably formulated as a spray and administered in an effective amount to a female subject(s) for inhalation by a male subject(s).

### BRIEF DESCRIPTION OF THE DRAWINGS

FIG. 1 is a graphical depiction of the interaction between perceived age and picture gender.

### DETAILED DESCRIPTION OF THE INVENTION

According to the method, a composition containing an odorant or odorant mixture is administered to a female subject for sniffing and inhalation by a male subject into the nasal passageway, to deliver an amount of the odorant or odorant mixture effective to alter the male subject's perception of the age of observed or viewed female individuals.

The composition containing the odorant or odorant mixture is formulated to cause an alteration in perception of the male individual inhaling the odorant/odorant mixture of the age of a female individual who is viewed by the male. Such an effect can be assessed and measured subjectively by interviewing and questioning the male individual about their perception of the age of a viewed female before and after the administration of the odorant, and assessing the male individual's response. One preferred odorant is an aromatic substance to which the inhaling male individual displays a negative hedonic response. A preferred composition for administration comprises a grapefruit odorant, preferably a pink grapefruit odorant, as the dominant odor or essence of the composition. In a preferred embodiment, the composition contains a mixture of pink grapefruit, vanilla, and baby powder odorants.

As used herein, the term “odorant” refers to an odor-causing chemical compound or mixture of compounds that, when delivered in a gaseous or aerosol medium, can stimulate olfactory and/or trigeminal chemoreceptors in the nasal cavity and cause a physiological or psychological response. A hedonically positive odorant or odorant mixture is one to which the individual has a pleasant or positive reaction to its scent. A hedonically negative odorant or odorant mixture is one to which the individual has a repulsive or negative reaction to its scent.

In a preferred embodiment, the subject male individual is presented with the composition containing a suprathreshold concentration (e.g., about 25-55 decismel units) of the odorant or odorant mixture that is not so high as to become an irritant (trigeminal), and generally inhales the odorant for about three seconds to about one minute, preferably at least about 20 seconds. Preferably, the level or concentration of the odorant or odorant mixture within the composition, and/or mode of administering the composition is sufficient to overcome competing or conflicting ambient odors that may act to nullify its effect.

It is preferred that the effect of the odorant/odorant mixture on the perception of a normosmic male subject who finds the odorant or odorant mixture to be hedonically negative provides a change in the male subject's perception of the chronological age of the viewed female individual such that there is an at least about 3% reduction to up to about 20% or greater reduction in the perceived chronological age of the female individual as compared to the actual chronological age.

For normosmics who consider the odorant or odorant mixture to be hedonically positive, the result is preferably a change in the male subject's perception of the chronological age of the viewed female individual such that there is an at least about 5% reduction to up to an about 25% or greater reduction in the perceived chronological age compared to the actual chronological age of the female individual.

According to the invention, it was found that the administration of an odorant composition comprising a grapefruit odorant, preferably a pink grapefruit odorant, as the dominant or principal odor or essence, will significantly alter the perception of a male individual who inhales the odorant composition of the chronological age of a female individual who is observed by the male individual. In particular, it was found that the inhalation of a hedonically negative odorant composition formulated with a grapefruit odorant, preferably a pink grapefruit odorant, as the primary odor or essence, can significantly alter the perception of male subject who inhales the composition such that the chronological age of a female subject as perceived by the male subject is lower than the actual chronological age by up to about 20%, or about 4-10 years less than the actual chronological age of the female subject, more typically about 5-7 years less.

The odorant or odorant mixture is provided as a formulated composition of a single essential odorant or a blend (mixture) of the essential odorants to cause the desired effect, and eliminates odorants that compete with or mask the effective odorant(s). The odorant or odorant blend composition can be administered in combination with an odorless carrier such as mineral oil or water, and odorless additives such as preservatives and the like. Preferably, the odorant composition provides a hedonically negative response by the inhaling individual.

The preferred odorant composition is a formulation that essentially comprises a hedonically negative grapefruit odorant and eliminates odorants that compete with the grapefruit odorant accords or notes to provide a full effect on the male individual inhaling the odorant mixture. Examples of grapefruit odorants include grapefruit oil, pink grapefruit oil, and the like. A grapefruit odorant, and other odorants for use in the present methods, are readily available as a liquid, essential oil, extract, or other form from a variety of commercial sources, including, for example, Energy Essentials, AromaTech, Inc. (Somerville, N.J.), Florasynth, Inc. (Teterboro, N.J.), International Flavors and Fragrances, Inc. (IFF; New York, N.Y.), among others.

In another preferred embodiment, the composition contains a mixture of pink grapefruit, vanilla, and baby powder odorants.

In one embodiment, odorants or odorant mixtures can be readily screened and assessed for negative hedonics and effectiveness in altering the perception of an inhaling male subject of an observed female individual's chronological age. For example, a composition containing an odorant or odorant mixture can be administered to a male individual who is questioned as to a positive or negative reaction to the pleasantness of the scent. The composition can then be administered to the male individual for inhalation to assess its effectiveness in modifying their perception of the age of an observed female individual to a reduced age. The composition can also be assessed for its effect in providing a positive effect on a male subject who inhales the odorant/mixture resulting in the male perceiving the age of a viewed female individual as being less than the actual age. The composition can also be assessed for its effect on alleviating and reducing the anxieties of a viewed female individual, which are associated with the female's negative feeling of being old or perceived as old, as a result of the positive effect of the odorant/mixture on the inhaling and viewing male subject(s). In addition, the composition can be evaluated for its effect on reducing anxiety and stress, and/or elevating a sense of well-being in a male subject(s) who inhales the composition, which can be manifested, for example, by a change in the male subject's perception of the age of a female subject(s) in terms of a reduction in age compared to the female's actual age.

For example, a method of screening a composition containing an odorant or a mixture of odorants for effectively altering a male subject's perception of age of female individual can comprise the steps of: administering a suprathreshold and non-irritant concentration of a composition consisting essentially of one or more odorants to a male subject for inhalation, having the inhaling male subject estimate the age of a viewed female subject, comparing the estimate of the age to actual age of the viewed female subject to provide a “difference value”, comparing the difference value to a “control value” to determine the statistical significance of the difference value, and eliminating the odorant or odorant mixture as being ineffective for altering the male subject's perception of age if not statistically significant. The control value can be derived by having the inhaling male subject estimate the age of the viewed female subject without inhaling the composition (or inhaling an odorless control composition), and comparing the age estimate with the age of the viewed female subject to provide the control value, preferably before administering the test composition to be screened.

The screening test as well as the method of the invention can include other steps such as having the inhaling male subject identify the composition as hedonically positive or hedonically negative, and testing olfactory ability and/or olfactory capacity of the male subject, among other olfactory tests known and used in the art.

According to the invention, a composition comprising the odorant or odorant mixture, e.g., a grapefruit odorant, is dispensed in an amount and time effective to provide a vaporous emission for inhalation by a male individual to effectively change the inhaling male individual's perception of the chronological age of a female subject. Such an effect can be assessed and measured subjectively by interviewing and questioning the inhaling male individual about their perception of age of the female subject before and after the administration of the odorant or odorant mixture, and assessing their response.

In one embodiment, the method of the invention can be used to alter the perception of a male subject (male observer) of the age of a female subject, particularly an elderly female individual, and thus increase the attractiveness and positive perception of the female subject to the male observer. This can, in turn, have secondary effects on the both the male subject and the female subject by enhancing a positive perception of the female subject. In such a method, a composition having an effective concentration of a suitable odorant or odorant mixture is administered to the observing male subject for inhalation for an effective time period such that the inhaling male subject perceives the age of a viewed female subject to be less than the female's actual chronological age, preferably lower by at least about 5%, more preferably by at least about 20% or greater, or preferably at least about 4-10 years less than the actual age, preferably at least 5-7 years less.

In one embodiment of the method, a composition having an effective concentration of an odorant or odorant mixture is administered to the observing male subject for inhalation, for example, by applying a composition comprising the odorant or odorant mixture to the body (e.g., skin) or clothing of a female subject by means of a dispensing device containing the odorant or odorant mixture (e.g., spray container, blister pack, cloth, etc.) such that the male subject can inhale the odorant/mixture in an amount effective to provide the desired effect on the male subject's perception of age of the female subject. In another embodiment, the odorant composition can be administered or presented to the male individual for inhalation, for example, by delivering the composition as a liquid to be inhaled within a container with a removable cap, as a spray onto the body of the male individual, as a “scratch-and-sniff” delivery system, in a blister pack containing the composition, and the like.

A composition comprising the odorant or odorant mixture is applied to the female individual to be viewed such that a sufficient concentration of the odorant or odorant mixture can be inhaled by the observing male subject to effect a change in the male subject's perception of the age of the observed or viewed female individual. The details of formulating and applying a suitable concentration of the odorant or mixed odorant composition to the female individual to be viewed to achieve that end are well within the skill of the art.

The odorant composition comprising the odorant or odorant mixture is dispensed to the inhaling male individual in a form that provides a vaporous emission for inhalation. For example, the odorant composition can be administered to a female individual, for example, by applying the composition to the female individual's skin in a number of forms including, for example, as a liquid, powder, gel, cream, paste, and the like. The odorant composition can be administered in combination with an odorless carrier such as mineral oil or water and/or odorless additives, and can be formulated with a viscosity effective to allow for aerosolization. The odorant composition can be dispensed onto the skin, for example, by direct application, by contact with a cloth carrying the composition, by an aerosol or nonaerosol spray, among other modes of application.

The odorant composition can also be inhaled from a device, for example, from a capped vessel containing a liquid or solid form of the odorant or mixture, the liquid form being optionally absorbed to a wicking material, from a blister pack or scratch-and-sniff odor patch containing microcapsules of the odorant, as a spray from an aerosol or non-aerosol pump-type spray device, as a nasal spray, by means of a scented cloth, and the like. It is preferred that the odorant composition is provided in a portable dispenser that is easily transportable and readily accessible.

The odorant composition can be packaged as part of an article of manufacture, or kit. In one embodiment, the article of manufacture can comprise a container of an odorant composition or, packaged together, a container of a first odorant and a container of a second odorant (etc.) for combining together to form the odorant composition. The odorant composition of the invention comprises a composition comprising an odorant or mixture of odorants in a suprathreshold and non-irritant concentration effective to alter a male subject's perception of a viewed female individual's chronological age upon inhalation such that the female's age is perceived as being less than the actual age. In one embodiment, the composition comprises one or more hedonically negative odorants. In a preferred embodiment, the composition consists essentially of a grapefruit odorant. For example, the article of manufacture can comprise a container of an odorant composition consisting essentially of a grapefruit odorant. Preferably, the composition comprises one or more odorants of which a grapefruit odorant is the dominant odor or essence, with a pink grapefruit odorant being preferred. A preferred composition contains a mixture of pink grapefruit, vanilla, and baby powder odorants.

Examples of containers for the composition include a vial, jar, pouch, can, bottle, cloth, aerosolizer, blister pack, a booklet of scratch-and-sniff odor patches, and the like. The article of manufacture can further comprise written or other format of instructions (e.g., C.D., video, cassette tapes, etc.) for use of the odorant composition for altering perception of age in a method according to the invention, including, but not limited to applications such as treating anxiety associated with age, enhancing self-esteem, treating mild depression associated with age, etc. In another embodiment, the article of manufacture can comprise packaging material and an odorant composition according to the invention contained within the packaging material, wherein the packaging material comprises a label that indicates that the odorant composition can be used for altering perception of a female's age, as an adjuvant for treating a patient for low self-esteem and/or mild depression relating to age, and/or alleviating a person's stress relating to age. The article of manufacture can also include an odorant composition and instructions for testing olfactory threshold according methods known in the art. The parts of the article of manufacturing can be contained or separately packaged within a packaging material, such as a box, bag, pouch, and the like.

An odorant is presented at a suprathreshold level when the decismel level or concentration of the odorant is beyond that needed to be detected by a normosmic individual. At its irritative level, the odorant quantity is so high and intense that the odorant stimulates predominantly the trigeminal nerve (for pain) rather than the olfactory nerve and, hence, is perceived as noxious or painful. The irritation threshold of the patient is the lowest concentration of the substance that causes immediate stinging or burning sensations in the nose, or stinging or lacrimation of the eye. (See, J. F. Gent, in Clinical Measurement of Taste and Smell, pages 107-166, H. L. Meiselman et al. (eds.), 602 pp., MacMillan, NY (1986); R. L. Doty et al., Ann. Neurol. 25: 166-171 (1989); E. Koss et al., Neurology 38: 1228-1232 (1988); and R. Doty, The Smell Identification Test: Administration Manual 1983: 13-14, Philadelphia: Sensonics, Inc. (1983)).

If desired, prior to the administration of the odorant, the male subject can undergo olfactory testing according to a test such as the University of Pennsylvania Smell Identification Test (UPSIT), a 40-question forced-choice, scratch-and-sniff identification test, and the Chicago Smell Test, a 3-item detection and identification test (R. Doty, The Smell Identification Test: Administration Manual 1983: 13-14, Philadelphia: Sensonics, Inc. (1983); A. R. Hirsch et al., Chemical Senses 18(5): 570-571 (1993); A. R. Hirsch et al., Chemical Senses 17(5): 643 (1992)).

The male subject can also be evaluated for olfactory capacity (e.g. loss of smell) according to an olfactory threshold test as known and used in the art. Such a test provides a precise magnitude of loss of smell and classifies the individual as normosmic, hyposmic or anosmic, which is useful in assessing the effectiveness of a particular odorant and/or the required concentration of the odorant to provide a suprathreshold level to effectively change perception of chronological age. According to that test, an odorant substance such as butyl alcohol, phenyl ethyl alcohol, or pyridine, is combined in an odorless liquid medium to provide a series of dilutions, or binary steps, of the odorant. For each successive binary step up the dilution scale, the odorant is present, for example, at one half the concentration of the preceding step. The highest concentration of the odorant usually provides the substance at an irritant level. The individual is presented with the series of dilutions in ascending order, and is asked to compare each dilution step to at least one control stimulus, such as odorless propylene glycol.

Ranges of the average normal threshold for various odorant substances can be found in the art, for example, Amoore and O'Neill, “Proposal for Unifying Scale to Express Olfactory Thresholds and Odor Levels: The “Decismel Scale”,” in Proceedings of the 1988 Air Pollution control Association Annual Meeting, Paper No. 78.5 (21 pp.), Air and Waste Management Association, Pittsburgh, Pa. (1988); Amoore and Haotala, “Odor as an Aid to Chemical Safety: Odor Thresholds Compared with Threshold Limit Values and Volatiles for 214 Industrial Chemicals in Air and Water Dilution,” J. Appl. Toxicology 3(6):272-290 (1983).

A suprathreshold amount is a concentration of the odorant/odorant mixture is greater than the threshold amount. The normal threshold concentration can be determined by administering a series of the same concentrations of the odorant/odorant mixture to a control group of at least 25 individuals who do not have a chemosensory dysfunction, and calculating the mean threshold concentration detected by the group of 25 individuals. Another alternative is to refer to the known threshold concentration value for the odorant/odorant mixture that has been established previously and published by J. Amoore et al., J. Appl. Toxicology, 3:272 (1983).

Odor thresholds can be expressed on the decismel scale. The decismel scale is constructed by setting the mean threshold concentration of a chemosensory agent detected by the control group of 20 year olds at the 0 value. A decismel is calculated by dividing the concentration of the odorant detected by the patient by the normal threshold concentration (using the published value or empirically determining the value) and then taking the logarithm of the quotient. The logarithm of the quotient is then multiplied by 20 to obtain the decismel value. Decismel values can be positive or negative. A positive decismel value indicates the patient is less sensitive to the odorant, i.e. has a higher threshold detection concentration. A negative decismel value indicates that the patient is more sensitive to the compound, i.e. has a lower threshold detection concentration. An increase in the threshold concentration value over the mean threshold concentration value of 2-fold corresponds to 6 decismels (or ds). Determination of decismel units is known in the art, as addressed, for example, in U.S. Pat. Nos. 5,380,765 and 5,492,934 (Hirsch).

In the art, a “normosmic” individual is one who can detect the odor of a substance without irritant sensations when the odorant is presented with the range of its average normal threshold. A “hyposmic” or “microsmic” individual has reduced capacity of the olfactory nerve being able to detect an odorant substance by its odor at a concentration, or decismel level, above that of a normosmic individual yet below its irritant concentration level. An “anosmic” individual is one who has essentially no olfactory nerve capacity being unable to detect the odor of the odorant substance, but has trigeminal nerve function, being able to detect an odorant substance by means of irritant, tingling sensations when it is present at an irritant concentration. A patient who is able to detect pyridine vapor by means of irritant, tingling sensations caused by stimulation of the trigeminal nerve, but who cannot distinguish a pyridine odor at a lower concentration without such sensation, is considered to be anosmic having no olfactory nerve sensitivity.

The invention will be further described by reference to the following detailed example. This example is not meant to limit the scope of the invention that has been set forth in the foregoing description. Variation within the concepts of the invention is apparent to those skilled in the art. The disclosures of the cited references throughout the application are incorporated by reference herein.

### EXAMPLE 1

Test Study. The following study was conducted to assess the effect of three odorants to induce a change in an individual's perception of the chronological age of another (viewed) individual.

Methods

In a randomized, single blinded, counter-balanced order, two sets of ten pictures were presented to thirty-seven subjects while wearing surgical masks having been impregnated with a variety of different odorants and a blank, unodorized mask. After wearing the mask for one (1) minute (to account for novelty effect) (20, 21) subjects were presented with a consecutive sequence of ten (10) black and white portraits from 108 Portraits (22), for which they were requested to estimate an age. The mask was removed and a questionnaire using a ten-point analog scale assessing the hedonic nature of the mask's odor and perception of the smell were completed. Then subjects underwent a no-odor, no mask, three-minute wash out interval. The above procedure was repeated with the opposite mask. Five additional subjects were tested with two blank masks and no odorant to access testing variability and protocol technique. Seventeen people were tested with the grape aroma, four people were tested with the cucumber aroma, and sixteen people were tested with the pink grapefruit aroma. After completing the hedonics form on the second mask, expectancy effect (23) and placebo bias (24) was ascertained by asking the following questions: (1) Did you expect the odor would do anything to perceived age? (2) Do you believe in aromatherapy? (3) Do you use aromatherapy?

Subjects filled out a demographic questionnaire and were quarried regarding self-perception of olfactory ability and hedonics for each odor tested.

Total estimated ages for the odorized series was compared to the total estimated ages for the unodorized series. Statistical correlation was determined using a paired T-test, with p<0.05 delineated to be statistically significant. Analysis was also assessed independently for each subject's assessment of male or female model photographs as well as for sex of subject.

Data was analyzed for significant differences of average change of perceived age with odorant as opposed to blank. This was segregated based on hedonics, belief in aromatherapy, expectation of effect, and order of presentation (blank or odorant first).

Results

Of the five additional subjects who underwent double blank masks, no significant difference between trials was noted (p=0.567). No statistically significant effect of the grape aroma (p=0.198) and the cucumber aroma (p=0.244) was seen. The pink grapefruit aroma was from AromaTech (#236927).

Grapefruit trial. There were eight male subjects and eight female subjects in the grapefruit trial. The subjects were Asian, married and perceived themselves to have a normal sense of smell. The average age (standard deviation) was 43.0 years (8.28) (ranging from 35 to 71) for these subjects, 41.6 years (4.93) (ranging from 35 to 50) for men, and 44.4 years (10.88) (ranging from 39 to 71) for women. The mean estimation of age for each picture group was calculated by subject, and the age difference between blank and odorized masks within each subject was calculated.

Two-sided T tests with a p value less than a 0.05 significance level were performed for the differences between blank and pink grapefruit odorized masks for overall subjects, and for each gender. Overall, the difference between blank and pink grapefruit odorized masks was 2.69 years (p=0.025). The differences for male and female pictures were 0.45 years (p=0.247) and 4.93 years (p=0.053), respectively.

Stratification analysis was conducted by gender. For male subjects, the difference between blank and pink grapefruit odorized masks was 1.78 years (p=0.091) for all pictures, and the differences for male and female pictures were 0.65 years (p=0. 154) and 2.90 years (p=0. 155), respectively. For female subjects, the difference between blank and pink grapefruit odorized masks was 3.60 years (p=0.164) for all pictures, and the differences for male and female pictures were 6.10 years (p=0.891) and 0.25 years (p=0.641), respectively.

Analysis was also performed for odor hedonics and expectation effect of odor on perceived age. “Hedonically positive” was defined as a subject rating of the pink grapefruit smell as positive on a forced choice scale of either positive (+) or negative (−). “Hedonically negative” was defined as a subject rating of the pink grapefruit smell as negative on a forced choice scale of either positive (+), neutral, or negative (−). In the six subjects who rated the pink grapefruit aroma as hedonically positive (i.e., greater than neutral), the overall difference between blank and pink grapefruit odorized masks was not statistically significant at 3.13 years (p=0.2 11). In those who viewed the aroma in a hedonically positive manner, there were no statistically significant differences for male and females pictures: 0.00 years (p=0.977) and 6.27 years (p=0.158), respectively. However, in the ten subjects who rated the odor hedonically negative, the overall difference between blank and pink grapefruit odorized masks was a statistically significant 2.42 years (p=0.007). The differences for male and female pictures were not statistically significant at 0.72 (p=0.139) and 4.12 (p=0.291) years, respectively.

The definition of “perceived age” was based on the answer for question “Did you expect the odor would do anything to perceived age? Yes/No” Overall in the grapefruit study, 37.5% thought it would have a positive effect on age perception. In the six subjects who felt that aromas would affect perceived age, no statistically significant effect was seen. The overall difference between blank and pink grapefruit odorized masks was 3.20 years (p=0.091) and the differences for male and female pictures were −2.17 (p=0.415) and 8.57 (p=0.085) years, respectively. Likewise in the ten subjects who did not feel aromas would affect perceived age, no statistically significant effect was present. The overall difference between blank and pink grapefruit odorized masks was 2.38 years (p=0.116) and the differences for male and female pictures were 2.02 (p=0.088) and 2.74 (p=0.347) years, respectively.

The definition of aromatherapy belief was based on the answer to the question: “Do you believe in aromatherapy? Yes/No” . Overall in the grapefruit study, twelve subjects had a positive inclination towards aromatherapy. In the twelve subjects who believed in aromatherapy, a significant effect was seen. The overall difference between blank and pink grapefruit odorized masks was 2.85 years (p=0.045). The differences for male and female pictures were 0.48 (p=0.418) and 5.22 (p=0.057) years, respectively. In the four subjects who did not believe in aromatherapy, a significant change was not observed. The overall difference between blank and pink grapefruit odorized masks was 2.38 years (p=0.401). The differences for male and female pictures were 0.35 (p=0.233) and 4.05 (p=0.623) years, respectively.

The results are summarized in Table 1 (below).

TABLE 1
Differences [1] in Age Estimation between Blank and Perfume Masks

| Population | (N) | Overall Pictures | Male Pictures | Female Pictures |
|---|---|---|---|---|
| All Subjects | (16) | 2.69 (p = 0.025) | 0.45 (p = 0.247) | 4.93 (p = 0.053) |
| Male Subjects | (8) | 1.78 (p = 0.096) | 0.65 (p = 0.154) | 2.90 (p = 0.185) |
| Female Subjects | (8) | 3.60 (p = 0.164) | 0.25 (p = 0.641) | 6.95 (p = 0.177) |
| Viewed Odor as Hedonic | (6) | 3.13 (p = 0.310) | 0.00 (p = 0.977) | 6.27 (p = 0.118) |
| Positive | | | | |
| Viewed Odor as Hedonic | (10) | 2.42 (p = 0.007) | 0.72 (p = 0.139) | 4.12 (p = 0.291) |
| Negative | | | | |
| Belief that Odor Will Affect | (6) | 3.20 (p = 0.091) | −2.17 (p = 0.415) | 8.57 (p = 0.085) |
| Perceived Age | | | | |
| Belief that Odor Will Not | (10) | 2.38 (p = 0.112) | 2.02 (p = 0.088) | 2.74 (p = 0.347) |
| Affect Perceived Age | | | | |
| Belief in Aromatherapy | (12) | 2.85 (p = 0.045) | 0.48 (p = 0.418) | 5.22 (p = 0.057) |
| No Belief in Aromatherapy | (4) | 2.38 (p = 0.401) | 0.35 (p = 0.233) | 4.05 (p = 0.623) |

[1] Difference (years) = Blank − Perfume

Since the study was designed as a two-treatment (odorized and nonodorized masks) and two-period (which mask was presented first) crossover (order) study, possible crossover (order) effect was evaluated for the age estimation between blank and grapefruit masks. There was no statistically significant crossover (order) effect at two-sided T-test p value>0.100. A possible crossover (order) effect (p=0.040) on age estimation for female pictures 10 was observed in those who viewed the odor as hedonically negative. Further analysis of the hedonically negative subgroups was conducted to exclude the possible crossover effect from the first set of photographs using the data collected. The results were consistent with the previous observation from both the first and second set of photographs. Controlling for the crossover effect, the rejuvenating effect (4.23 years) was significantly greater in overall subjects with odorized masks (p=0.028).

The interaction between each variable of interest (subject gender, hedonic positive, belief that odor will effect perceived age, and belief in aromatherapy) and picture gender was evaluated with a two-sided T-test with p value as less than a 0.05 level designated as being significant. Only one result met statistical significance: expectation of odor with perception of age and differential effect on picture gender (p=0.032). Thus, in those who did believe odor had an impact on perception of age, photographs of women were perceived to be younger.

The pink grapefruit aroma effected people's age estimations. Subjects wearing masks impregnated with the pink grapefruit aroma provided younger age estimates than when wearing a non-odorized mask. For all subjects, the age difference between blank and perfume masks were 2.69 years (p=0.025) for all pictures, 0.45 years (p=0.247) for pictures of men, and 4.93 years (p=0.053) for pictures of women. The differences were statistically significant for overall pictures. The age difference between blank and perfume mask was larger in female subjects for overall pictures (female subjects: 3.60 years vs. male subjects: 1.78 years), and for female pictures (female subjects: 6.95 years vs. male subjects: 2.90 years). The age difference between blank and perfume mask was similar between female and male subjects for male pictures (female subjects: 0.25 years vs. male subjects: 0.65 years). There was a possible interaction between gender and aroma (perfume). For both genders, the rejuvenating effect on female pictures was greater than that on male pictures. In male subjects, the rejuvenating effect for female and male pictures were not as great as that seen in female subjects (male subjects: 2.25 years (=2.90−0.65), female subjects: 6.70 years (=6.95−0.25)).

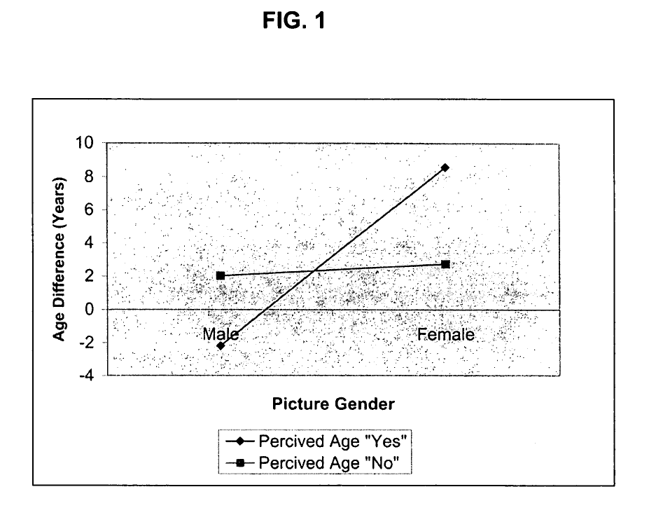

Since a statistically significant interaction between the data from those who believed odors would affect perceived age and picture gender was observed (p=0.032); subjects who thought that the odor would impact on the estimation of perceived age tended to provide younger age estimation in response to the grapefruit aroma for female pictures compared to those who did not. This demonstrates expectancy effect. See FIG. 1.

Overall, this study found that odor may affect people's estimation of age, the pink grapefruit odor caused subjects to yield younger age estimates, and the rejuvenating effect of odor had differential effects based on gender.

Discussion

It was found that inhaling the aroma of pink grapefruit induced a lower perception in the inhaling individual of the age of an observed individual.

Odorants have been found to affect perception of different aspects of photographs and videos, including level of attractiveness (25, 71) and weight (19). More specifically, pyridine has been demonstrated to increase sensitivity of observers to light. (26) Reduction of perception of red and violet colors and enhancement of perception of green color has been shown with the oil of geranium. (27)

Although not intended to be a limitation to the present disclosure, there are several potential mechanisms whereby the pink grapefruit aroma reduced age estimation.

The aroma may have induced a change in mood state causing the subjects to be happier, and in a happier mood, people and objects are judged in a more positive or optimistic light. (28, 77) Since youth is considered as a positive attribute, this manifests as a lower estimation of age. Thus, pink grapefruit may have generated its rejuvenating effects through the process of affect infusion, whereby affect impacts upon cognitive processes (86).

It is doubtful that this is a primary mechanism of pink grapefruit's effect on age perception since in those who had negative hedonics for pink grapefruit aroma, there was an even greater rejuvenating effect than was found in those who viewed it as hedonically positive. Smelling hedonically positive odors typically induces a positive affect, and a hedonically negative odor typically induces a negative affect. (29, 30, 31, 36, 49) That grapefruit's effect was most marked in those with negative hedonics implies that sadness rather than happiness was induced, but still, a rejuvenating effect was seen. However, this incongruency between presumed affect generated and perception of the models does not rule out that affect modulation was the primary rejuvenating action. Negative affect has been demonstrated to facilitate greater motivation and efficacy in decision makers and greater motivation may have caused subjects to lower their estimation of age (87).

Alternatively, the grapefruit aroma may have induced an olfactory-evoked nostalgic effect. The aroma may have induced a memory of childhood, which are usually fond memories associated with positive feelings and happiness. (32, 33) These happy memories act to uplift mood (29), again positively biasing the observer in favor of the subjects of the photos, with attendant reduction in estimation of age.

Possibly, the odors may have acted on actual visual perception or associations, thus age estimation. This could be understood through a direct affect of grapefruit odor on recognition of visual stimuli or effects of odor on occipital lobe association fibers. (34) The impact of one special sensory system on another has been demonstrated with odorants interacting on simple visual stimuli (color, smell) (35, 37) as well as on complex visual perception integration tasks (estimation of weight). (19) Thus, the aroma could have had its impact through induction of visual distortion—the hyperfunctional lines could have been viewed as more shallow, reducing the perception of age (3, 4, 5, 6, 7, 8), similar to the perceived rejuvenating mechanisms of botulism toxin. (4) A similar mechanism of action of odor on visual perception has been proposed with regards to perception of size of external space. (18)

Another possible mechanism is that the grapefruit aroma could have sexually aroused the subjects thus clouding their judgments. This could have caused the subjects to be more generous in their appreciation of the desired sex, as manifested by the downward estimation of age. While the current study did not include assessment of sexual arousal, Hirsch et al. have demonstrated that odors can impact arousals of females and males, specifically that the aroma of pink grapefruit increases penile blood flow by 25%. (14, 15, 16)

Alternatively, the grapefruit aroma may have acted as an anxiolytic. (31, 36, 76, 80) Stress removal induces euphoria or happiness. By reducing baseline stress, a happy state occurs (38, 39, 40), thus providing the backdrop for a more optimistic or enlightened view of the subjects' photographs. Thus, the demonstrated effect may be due to the aroma's anxiolytic nature, which is also postulated to be the mechanism of odor on perception of external space.(18)

Possibly, the grapefruit aroma acted to induce the subjects to be in a more receptive state and more positively inclined towards the people in the photographs. The effects of odor on acceptance of people and ideas have been demonstrated in relationship to other food odorants. (58) The aroma of garlic bread presented concomitantly with a pasta meal enhanced positive interaction by 8% and decreased by 22% negative interactions. (42)

Congruency between odorant, the model, and expectation should be considered. The importance of congruency of odors has been demonstrated in the evaluation of the quality of inanimate objects. (41, 43, 45) The scent of an object itself becomes an important attribute of a product and thus becomes a part of consumer expectations. (61) Consumers prefer products with an appropriate smell to those with an inappropriate smell. (44) Also, artwork is more appreciated and enjoyed as higher quality when viewed in presence of an odor perceived to be congruent to the art. (59) It is possible that the grapefruit odor presented concurrently with photographs of the models were congruent with the subjects' expectations of what the model should smell like. By meeting the subjects' expectations, they would feel validated, inducing a more positive mood state, which would then act to enhance a positive perception of the model as measured by age. On the other hand, viewing model's photographs while smelling odorants, which were not congruent with expectations, would tend to cause subjects to view the models in a negative light. This negative perception would then be reflected by a greater estimate of perceived age.

It is possible that the expression of the models when photographed could have influenced results. This could be understood given the expectation of congruence of odor with affect as displayed by the model's facial expression. (38, 46) As in The Invalid story by Mark Twain, (60) the perception of the source of aromas affects the aromas' hedonics. (37) Thus, if the photo displayed negative affect in facial expression, the otherwise pleasant odor may have been perceived as unpleasant. The hedonically unpleasant odor would have been congruent with the expectations based on the negative facial expressions. Odor hedonics matching emotions as manifested by facial expressions would thus induce a positive mood state since expectations had been met. This positive mood would be transferred to the perception of the model, which would be viewed positively and thus younger. An unpleasant facial expression amongst models in the photographs would thus be congruent with the hedonically negative odor, and meeting subjects' expectations and inducing a positive mood with associated reduction in age estimation. This would thus explain our findings of an inverse correlation between odor hedonics and rejuvenating effect. (36, 62)

Alternatively, the congruence between expectation and aroma may not be for the expectations given the appearance of the model but rather for subject's expectations of what a rejuvenating aroma should smell like. Pink grapefruit aroma may have been an anticipated rejuvenating aroma. If so, presentation of this odor would meet subject's expectations, inducing a sense of self validation and confidence in the subjects, and a greater self esteem and happiness. And happiness is associated with a more optimistic and friendly outlook. This positive feeling could promote a change in the perception of age.

Several potential limitations in the present study should be addressed. One such limitation of this study was that odorants were used to assess the age of individuals in photographs, and not the actual person in a natural setting. The question thus arises as to whether non-visual stimuli would have an impact upon perception of age (i.e. gait, actions, etc.), minimizing the effect of the aroma. However, clinical data tends to discount this on the basis that visual stimuli are the primary mediators of age perception. (3, 8, 47)

Expectancy effect should also be considered. (23) This is the phenomenon whereby because the individual believes that the aroma will have an effect on their responses, it does. This expectancy effect has been demonstrated with effects of odors on trigeminal sensations (23) and contingent negative variation. (53) We anticipated that this might occur in our study, and controlled for expectancy effects by querying about orientation towards aromatherapy. We assumed that if a subject believed in aromatherapy, there would be a positive expectancy effect; if they did not believe in aromatherapy, there would be a neutral or negative expectancy effect.

To even further compensate for expectancy effect, subjects were queried specifically about their belief as to whether aromas may effect age perception. While we did not find any expectancy effect based on belief as to whether an odor can affect age perception, we did find an effect based on belief in aromatherapy.

The sequence of the presentation of blank/odorized mask was randomized to control for the effects of the order of presentation of odorants stimulation. However, an order effect was still noted. While the protocol required a three-minute delay between masks, the effect of the grapefruit aroma may have lingered beyond these three minutes.

Sex or gender difference was also anticipated. In other studies, odors have had a greater effect on women in purchases and perception of value of inanimate objects (44, 45), claustrophobia (18), learning (17), and migraine (57), than in men. This gender difference was not observed. While when stratified for gender, sex differences were not statistically significant, the grapefruit aroma had a greater rejuvenating effect with females as opposed to male subjects (3.60 vs. 1.78 years).

Because odor on weight perception impacted men but not women, (19) we also subdivided the data by sex of subjects and sex of photographs. These results demonstrated the pink grapefruit aroma reduced the perception of women's age by five years, but it did not affect perception of men's age.

The hedonics of the grapefruit odor was indicated as a primary mediator of its effect. In those with negative hedonics, we predicted that there would not have an age-reducing effect. If this were seen, it would imply that the mechanism of the odor would have been an action mediated through the limbic system with an induction of positive affect resulting in affect infusion (86). The same hedonic dependent action has been seen with a variety of effect of aromas including claustrophobia (18), migraine (57), and learning ability (17). However, we did not find a correlation between hedonics and reduced perception of age. Rather, we found just the opposite—inverse correlation between odor hedonics and reduction of perceived age. These findings contradict studies, which suggest that malodors detract from attractiveness. (95) Although not readily explainable, the results are consistent with findings of greater camaraderie and positive feelings to others in a group, who have all been exposed to a malodor (49).

Another factor that was considered is the intensity of the aroma that was administered. The higher the intensity, the more negative the hedonics is perceived (50, 51, 52, 75). Therefore, if the intensity of the aroma in the mask were too strong, the affect would tend to be negative. However, this was not likely a factor because the levels tested were judged as “not too high an intensity” by the panel at The Smell and Taste Treatment and Research Foundation, Chicago, Ill.

Another factor was the source of the aroma. In the case of a live model, the aroma would be from the model. Under the test conditions, the aroma was from the facemask. This may have set up an artificial situation. In the presence of a more natural environment, a rejuvenating effect may have been of a different magnitude.

The subjects studied were Asian American, which may have influenced the results based on the subjects' cultural background. Olfactory ability has been shown to be influenced upon ethnic background. In the United States, Korean Americans (54) have greater olfactory senses than others, while native Japanese have the worst olfactory ability. Furthermore, different cultures have different olfactory hedonic parameters. (55)

Another consideration is demographic factors. Women have demonstrated a sharper sense of smell than do men, especially at the time of ovulation. (63) Intrinsic olfactory abilities can modulate any influence of an aroma on age perception. For example, factors that can decrease olfactory ability include sinusitis (64, 56), allergic rhinitis (65), hypothyroidism (66), diabetes mellitus (48), and AIDS (67). Not only can diseases cause a decline in olfactory ability, environmental pollution and harmful poisons (68) including sulfur dioxide, and an assortment of other toxins as a result of primary and secondary exposure to cigarette smoke, can also impair olfaction. (68) As a result of such impaired olfactory abilities, otherwise pleasant odors may be converted to hedonically negative. This exposure to a hedonically negative odorant may envelope a negative aura to the persona of the picture, thus interfering with the rejuvenating effects of the aroma.

The subject's preset notion associating different aromas with different generations may be one mechanism of action. Studies of olfactory evoked nostalgia have shown that different generations have nostalgic memories for distinct aromas: younger generations for artificial aromas, older generations for more naturalistic smells. (33) Thus, introducing an aroma specific to a generation may cause subjects to associate this aroma with the associated age of the generation, causing them to project this age onto the perceived age of the models.

The subject's past experience with an aroma and the person in the subject's life who wore the aroma may impact age perception (69). The grapefruit aroma may have acted as a rejuvenator through the mechanism of context dependent learning (81, 84). For instance, a subject whose grandmother often wore lavender may consciously or subconsciously associate lavender with octogenarians, whereas the aroma of cotton candy may remind them of more youthful acquaintances. Thus, the aromas, which are traditionally used with older individuals, would act to increase the perceived age where traditionally more youthful smells would serve to induce a rejuvenating effect.

Alternatively, in the presence of the pink grapefruit aroma, the subjects may have felt better about themselves. When one feels more confident, there is less of a need to degrade others, and ageism is one form of degradation. Enhanced self-confidence may reduce negative affect and thus perceived age of the models would be lowered. This mechanism implies that grapefruit aroma may promote self-confidence and enhance self-esteem. As such, the administration of a grapefruit aroma would have efficacy in treating social phobia or low self-esteem.

The impact of the subject's age may also have had an impact on age perception. Younger people have better sense of smell than older people (70, 78, 79). Thus, the grapefruit aroma may have a greater impact of perception of age in younger subjects. However, younger and older people may estimate age in different ways, since older people have had more experience estimating age based both on their own reality of the vicissitudes of life as viewed from their reflection of their own self-image and through more years of experience of estimating age than young people. Thus in the elderly, we would anticipate a greater accuracy of age perception based on visual stimuli since the concept of age would be less ambiguous. This highlights the concept of the ambiguous nature of the variable attempting to be detected. If the variable is less ambiguous, one may expect ambient odor to have less of an impact. On the other hand, the more ambiguous the attribute to be assessed, the greater impact the odor will have. Thus, while an odor would not influence processes which require distinct and well-defined cognitive components such as calculations, it would have an effect on more poorly delineated, less logically required decisions, such as level of attraction on perception of weight (19).

Likewise, since odorants are processed in the non-dominant hemisphere, where the affect is localized, the inhalation of aromas would affect factors that are more emotionally-based than cognitively-based (82). Since ageism is imbued with affect, it is understandable that aromas would effect age estimations more than calculations, for instance, because logically reasoned mathematics are localized in the dominant hemisphere with logical reasoning and odor preferential influence the non-dominant hemisphere.

The aromas may have had differential impact upon age perception based on the authoritative countenance of the models. People generally tend to view authorities as being older. Those less authoritative and more jovial are viewed as being younger. The pose and facial expression of the models thus may have influenced the efficacy of the odors tested.

Another possible alternative is that the aroma made the subject feel more connected to the models. In other studies, the presence of an unpleasant aroma created a bonding between subjects and models who were forced to endure common malodor. Thus, the aromas made the subject feel more connected, and in our study, this may have induced a subtle linkage with the subjects and models. This bonding would then subconsciously induce a bonding and connection between the models whose pictures were viewed concomitantly with the malodor. This bonding would induce sympathy or positive views of the model causing the subjects to rate them in either a more positive (younger) manner or a manner most consistent with their own personal traits. This could explain why those who viewed the pink grapefruit odor hedonically negative perceived the models to be younger.

Alternatively, the odors may have impacted cognitively mediated processes rather than emotionally mediated processes, as in distraction. (37, 83) Odors can be demonstrated to influence these processes including learning (17), memory (32, 33), and weight perception (19). If estimation of age is based on a largely cognitive schema (i.e., hyperfunctional lines), this schema can be distorted or influenced by using odors. Therefore, by disrupting the central processes, odors could act to confuse and thus distort perception of age.

Although a small sample size was used in the present study, a statistically significant effect was observed. A logical extension would be to assess the effect of this aroma on subjects using a larger population size and different population demographics.

Given the strong prejudice against the aged, a modality that reduces the perception of age would have great social value. One of the benefits of administering aromas to reduce the perception of age is to reduce perceived age differences and minimize ageism. If younger people view older people as less old, they are more inclined to positively accept them.

In summary, while the determination of age has been primarily mediated visually, certain odors were determined to have an influence. In a trial, thirty-seven subjects (age average of 28 years, ranging from 13 years to 71 years) in a single blinded, randomized fashion, estimated the age of models in twenty photographs while wearing either blank masks or masks impregnated with a grape, cucumber or grapefruit aroma. The grape odor (p=0.198) and the cucumber odor (p=0.244) had no significant effects. The grapefruit aroma reduced subjects' perception of overall models' ages by an average of approximately three years (p=0.025) and of female models' age by five years (p=0.053). Possible mechanisms whereby the grapefruit aroma created a rejuvenating effect with relationship to perceived age includes induction of positive affect, sexual arousal, anxiolysis, change in cognitive set, and odor induced visual distortion. The perceived rejuvenating effect of the grapefruit aroma has potential utility in facilitating intergenerational communication or act as an adjuvant like botulism toxin, in cosmetic and pharmaceutical usage to impact perception of age.

Conclusion

Pink grapefruit aroma was observed to have a rejuvenating effect on perception of age. When viewed with this odor, models were estimated to be as much as three (3) years younger when compared with estimations of age without an odor present. While the pink grapefruit aroma did not statistically reduce the perceived age of male models, women were perceived to be five (5) years younger. Although not meant to limit the disclosure, the rejuvenating effect of the pink grapefruit aroma can be explained by considering the impact of odors on visual perception, sexual arousal, congruency effects, mood, facial expressions of the models, expectations, gender of the models, hedonics of the odor, and how age perception mechanisms, odor and affect are anatomically processed. These findings indicate a beneficial odor impact on enhancing intergenerational acceptance in our ageist society.

### EXAMPLE 2

Methods. Thirty-seven (37) subjects (age average of 28 years, ranging from 13 to 71 years) in a single blinded, randomized fashion, estimated the age of models in twenty (20) photographs while wearing either blank masks (no odor) or masks impregnated with a grape, cucumber, or grapefruit aroma.

Results. On average, the men in the study underestimated the age of the women in the photos by five years.

The grape odor (p=0.198) and the cucumber odor (p=0.244) had no significant effects. The grapefruit aroma reduced subjects' perception of overall models' ages by an average of approximately three years (p=0.025) and of female models' age by five years (p=0.053).

Conclusion. Only the grapefruit aroma had a rejuvenating effect on perception of age. In the women subjects, the inhalation of the grapefruit aroma did not have a significant effect on altering the women subject's perception of a man's age or of another women's age. In men, inhalation of the grapefruit aroma did not reduce the perceived age of other men, but women were perceived to be six (6) years younger.

Possible mechanisms whereby the grapefruit aroma created a rejuvenating effect with relationship to perceived age include induction of positive affect, sexual arousal, anxiolysis, change in cognitive set, and odor-induced visual distortion.

Although not meant to limit the scope of the disclosure or the invention, it is believed that, in men, the grapefruit aroma leads to heightened well being, which leads to a more “optimistic” view of the physical attributes of the women around them. It is further believed that the odor composition may act on the nondominant parietal lobe—the part of the brain used in integrating perception of age.

The perceived rejuvenating effect of the grapefruit aroma can be used to facilitate intergenerational communication or act as an adjuvant-like botulism toxin, in cosmetic and pharmaceutical usage to impact perception of age.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents. The disclosures of the cited patents, applications, and other references throughout the application are incorporated by reference herein.

### REFERENCES

1. Flemming, K. C., Evans, J. E., Chutka, D. S., “A Cultural and Economic History of Old Age in America.” Mayo Clinic Proc, 2003, Vol. 78, Pg: 914-921
2. Tefferi, A., LaRusso, N. F., “The “Age” Factor in Adult Medicine.” Mayo Clinic Proc, Copyright: 2003, Vol. 78, Pg: 913.
3. Osborn, D. R., “Beauty is as Beauty Does? Makeup and Posture Effects on Physical Attractiveness Judgments.” Journal of Applied Social Psychology, 1996, Vol. 26. Pg: 31-51.
4. Cash, T. F., Pruzinsky, T., “Body Image.” The Guilford Press, Copyright: 2002, New York. Pg: 84.
5. Baron, R. A., “Psychology”, Allyn and Bacon, Published 1997, Boston. Pg: 359-360.
6. Croft, J., “The Doctors Book of Home Remedies for Men”, Bantam Books, 2000, New York. Pg: 794.
7. Goldberg, K. A., “Longevity Program.” Rodale Inc. Press, Copyright 2001, New York. Pg: 257.
8. Charles, F. J., Cox, S., Earl, M., “Social Implications of Hyperfunctional Facial Line.” Dermatological Surgery: Official Publication for American Society for Dermatological Surgery [et al.]. May, 29, 2003 (5), Pg: 450-455.
9. Ralston, J. V., Fidas, G., Agli, C. E., Coppola, C., “Prosody as a Potential Cue For Age Perception.” ASA 131 s Meeting. May 1996.
10. Tranmiiler, H. Eriksson, A., Menard, L., “Perception of Speaker Age, Sex, and Vowel Quality Investigated Using a Stimuli Produced Within an Articulatory Model.”
11. Fischer, D. H., Growing Old in America, 1978, Pg: 77-102, 174, 208, 228.
12. Hirsch, A. R., “Effects of Ambient Odors on Slot Machine Usage in a Las Vegas Casino.” Psychology and Marketing, 1995, Vol. 12, No. 7. Pg: 585-594.
13. Hirsch, A. R., “Weight Reduction Through Inhalation of Odorants.” J. Neurol. Orthop. Med. Surg. Vol. 16. Pg: 28-31.
14. Hirsch, A. R., “Scent and Sexual Arousal.” Medical Aspects of Human Sexuality. 1998. Vol. 1, No. 3. Pg: 9-12.
15. Hirsch, A. R., Schroder, M., Gruss, J., Bermele, C., Zagorski, D., “Scentsational Sex.” The International Journal of Aromatherapy. 1998-1999. Vol. 9, No. 2. Pg: 70-81.
16. Hirsch, A. R., Gruss, J. J., “Human Male Sexual Response to Olfactory Stimuli.” J. Neurol. Orthop. Med. Surg. 1999, Vol. 19, Pg: 14-19.
17. Hirsch, A. R., Johnston, L. H., “The Impact Upon Learning by Floral Odor.” Chemical Senses, Vol. 20. No. 1, 1995. Pg: 77-78.
18. Hirsch, A. R., Gruss, J. J., “Ambient Odors in the Treatment of Claustrophobia: A Pilot Study.” J. Neurol. Orthop. Med. Surg. 1998, Vol. 18. Pg: 114-121.
19. Hirsch, A. R., Aleen, E. T., Busse, A. M., Hoogeveen, J. R., “The Effects of Odor on Weight Perception.” Chemical Senses. 2003, Vol. 28, A21.
20. Katz, D. B., Rogers, R. F., Steinmetz, J. E., “Novel Factors Contributing to the Expression of Latent Inhibition.” Behav Neurosci. October 2002; 116(5): 824-36.
21. Aberg, C., Nillson, L. G., “Facilitation of Source Discrimination in the Novelty Effect.” Scand. J. Psychol. September 2001; 42(4): 349-57.
22. Van Sant, G.: “108 Portraits,” Twin Palms Publisher, Santa Fe, N. Mex., 1992.
23. Dalton, O., Wysicki, C. J., “The Nature and Duration of Adaptation Following Short Term Exposure to Acetone.” Am J Ind Med. 46: 111-121.
24. Kirk-Smith, M. D., Boothe, D. A., “The Effect of Five Odorants on Mood and the Judgment of Others.” Chemical Signals in Vertebrates. Mac Donald, D. W., Muller-Schwartz, D., Natynzcule, S.: 48-54. Oxford University Press, Oxford, 1990.
25. Corley, N., Raudenbush, B., “Effects of Odorant Administration on Ratings of Physical Attractiveness and Personality Characteristics.” ACHEM-XXIV, 2002, 114: 29.
26. London, I. D., “Research on Sensory Interaction in the Soviet Union.” Psychological Bulletin, 1954, 51, 531-568.
27. Allen, F., Schwartz, A. “The Effect of Stimulation of the Senses of Vision, Hearing, Taste and Smell Upon the Sensibility of Organs of Vision.” Journal of General Physiology, 1940, 24, 105-121.
28. Forgas, J. P., Bower, G. H., Krantz, S. E., “The Influence of Mood on Perceptions of Social Interaction.” Journal of Experimental Social Psychology. 20, 497-513. 1984.
29. Bower, G. H., “Mood and Memory.” American Psychologist. Vol. 36, No. 2, 129-148. 1981.
30. Hirsch, A. R., “Olfaction and Anxiety.” The Clinical Psychiatry Quarterly, 1993, 16(1), Pg: 4.
31. Warm, J. S., Dember, W. N., “Effects of Fragrance on Vigilance Performance and Stress.” Perfumer and Flavorist. Vol. 15, 1990. Pg: 15-18.
32. Hirsch, A. R., “Scents of Childhood—Sense of the Past: How Odors Can Evoke Reminiscences,” Chicago Medicine, 1995, Vol. 98, No. 10, Pg: 16-19.
33. Hirsch, A. R., “Nostalgia: A Neuropsychiatric Understanding.” Advances in Consumer Research. 1992. Volume 19: 390-395.
34. Lishman, A. L., “Organic Psychiatry.” Oxford: Blackwell Scientific Publications, 1978, Pg: 74-75.
35. Bornstein, W. “On the Functional Relations of the Sense Organs to One Another and to the Organism as a Whole.” Journal of General Psychology, 1936, 15, 117-131.
36. Alaoui-Ismaili O et al.: “Basic Emotions Evoked by Odorants: Comparison Between Autonomic Responses and Self-Evaluation. Physiol Behay. 62: 713-720, 1997.
37. Engen, T., “The Effect of Expectation on Judgments of Odor.” Acta Psychologica, 1972, 36, 450-458.
38. Kraut, R. E., “Social Presence, Facial Feedback and Emotion.” Journal of Personality and Social Psychology, 1982; 42(5): 853-863.
39. Warren C. B., Munteanu, M. A., Schwartz, G. E., Benaim C., Walter H. G., Leight R. S., Withycombe, D. A., Mookberjee, B. D., Trenkle, R. W., “Method of Causing the Reduction of Physiological and/or Subjecteive Reactivity to Stress in Humans Being Subjected to Stress Conditions.” U.S. Pat. No. 4,671,959, 1987.
40. Toni, S., Fukuda, H., Kanemoto, H., Miyanchi R., Hamauzu Y., Kawasaki, M., “Contigent Negative Variation (CMV) and the Psychological Effects of Odor.” In: Van Toiler S., Dodd, G. H., (eds.). Perfumery: Psychology and Biology of Fragrance. New York: Chapman and Hall, 1988; 107-120.
41. King, J. R., In: S. Van Toiler, G H Dodd (eds). “Perfumery: The Psychology and Biology of Fragrance.” London: Chapman and Hall, 1988.
42. Hirsch, A. R., “Effects of Garlic Bread on Family Interaction.” Psychosomatic Medicine, Vol. 62, No. 1, 2000, Pg: 607-608.
43. Knasko, S. C., “Ambient Odor and Shopping Behavior.” Chemical Senses, 1989; 14: Pg: 781: A94.
44. Hirsch, A. R., “Sensory Marketing.” The International Journal of Aromatherapy, Spring 1993, Vol. 5, No. 1.
45. Hirsch, A. R., Gay, S., “The Effect of Ambient Olfactory Stimuli on the Evaluation of a Common Consumer Product.” Chemical Senses, Vol. 16, No. 5, 1991. Pg: 535.
46. Nezlek, J. B., Shean, G. D., “Fragrance Use and Social Interaction.” In Compendium of Olfactory Research. Ed, A N Gilbert, Dubuque; Kedall/Hunt Publishing Company, 1995, Pg: 73-79
47. Goin, J. B., Goin, M. K., “Changing the Body: Psychological Effects of Plastic Surgery.” Baltimore: Waverly Press, Inc., 1981: Pg: 61-68.
48. Settle, R. G., “The Chemical Senses in Diabetes.” In: Smell and Taste in Health and Disease. Ed: Getchell, T. V., Doty, R. L., Bartoshuk, L. M., Snew, J. B., Raven Press, New York, 1991. Pg: 829-843.
49. Rotton, J., Barry, T., Frey, J., and Soler, E., “Air Pollution and Interpersonal Attraction.” J. Appl. Social Psychol. 8:57-71 (1978).
50. Braiichli, P., Riiegg, P. B., Etzweiler, F., Zeier, H., “Electrocortical and Autonomic Alteration by Administration of a Pleasant and an Unpleasant Odor.” Chem Senses, 20, 505-515. 1995.
51. Henion, K. E., “Odor Pleasantness and Intensity: A Single Dimension?” J. Exp. Psychol. 90, 275-279. 1971.
52. Richardson, J. T. E., Zucco, G. M., “Cognition and Olfaction: A Review.” American Psychological Association. Inc. Vol. 105, No. 3, 352-360. 1989.
53. Lorrig, T. S., Roberts, M., “Odor and Cognitive Alteration of the Contingent Negative Variation.” Chem Senses. 15(5): 537-545, 1990.
54. Wysocki, C. J., Pierce, J. D. Jr., Gilbert, A. N., “Geographic, Cross-Cultural, and Individual Variation in Human Olfaction.” In: Smell and Taste in Health and Disease. Ed: Getchell, T. V., Doty, R. L., Bartoshuk, L. M., Snew, J. B., Raven Press, New York, 1991.
55. Engen, T., Gilmore, M. M., Mair, R. G., “Odor Memory.” In: Smell and Taste in Health and Disease. Ed: Getchell, T. V., Doty, R. L., Bartoshuk, L. M., Snew, J. B., Raven Press, New York, 1991.
56. Tovee, M. J., Reinhardt, S., Emery, J. L., Cornelissen, P. L., “Optimum Body-Mass Index and Maximum Sexual Attractiveness.” Lancet, 1998; 352:548.
57. Hirsch, A. R., Kang, C., “The Effect of Inhaling Green Apple Fragrance to Reduce the Severity of Migraine: A Pilot Study.” Headache Q., Vol. 9, 1998, Pg: 159-163.
58. Janis, I. L., Kaye, D., Kirschner, P., “Facilitating Effects of “Eating-While-Reading” On Responsiveness to Persuasive Communications.” Journal of Personality and Social Pyschology. 1963, Vol. 1, No. 2, Pg: 181-186.
59. Knasko, S. C., “Lingering Time in a Museum in the Presence of Congruent and Incongruent Odors.” Chem Sense, 18, 581, 1993.
60. Clemens, S. L., (1882), “The Invalid's Story.” In C. Neider (Ed.), The Complete Short Stories of Mark Twain. Garden City, N.Y., International Collectors Library, 1957.
61. Bone, P. F., Jantrania, S., “Olfaction as a Cue for Product Quality.” Marketing Letters, 1992; 313: Pg: 289-296.
62. Nezlek, J. B., Shean, G. D., “Social Introduction of Fragrance Use: A Summary of Research Conducted by the Fragrance Research Fund.” Perfumer and Flavorist, 1990; 15(3): Pg: 43-45.
63. Doty, R. L., Newhouse, M. G., Azzalina, J. D., “Internal Consistency and Short-term Test-retest Reliability of the University of Pennsylvania Smell Identification Test.” Chem Senses. 1985; 10: 297-300.
64. Scott, A. E., Cain, W. S., Leonard, G., “Nasal/Sinus Diseases and Olfactory Loss at the Connecticut Chemosensory Clinical Research Center.” Chem Sen, 1989, 14: 745.
65. Apter, A. J., Mott, A. E., Cain, W. S., Spiro, J. D., Barwick, M. C., “Olfactory Loss and Allergic Rhinitis.” J. Allergy Clin Immunol, 1992; 90: 670-680.
66. Mackay-Sim, A., “Changes in Smell and Taste Function in Thyroid, Parathyroid and Adrenal Diseases.” In: Smell and Taste in Health and Disease. Ed: Getchell, T. V., Doty, R. L., Bartoshuk, L. M., Snew, J. B., Raven Press, New York, 1991.
67. Murphy, C., Doty, R. L, Duncan, H. J., “Clinical Disorders of Olfaction.” In: Handbook of Olfaction arid Gustation. Ed: Doty, R. L., Marcel Dekker, 2003.
68. Amoore, J. E., Effects of Chemical Exposure on Olfaction in Humans.” In: Barrow C S ed. Taxicology of the Nasal Passages. New York: Hempisphere; 1986:155-190.
69. Cann, A., Ross, D. A., “Olfactory Stimuli as Context Cues in Human Memory.” American Journal of Psychology. Spring, 1989, Vol. 102, No. 1, Pg: 99-102.
70. Tuorila, H., Koskinen, S., “Olfactory Identification and Detection Tests in the Prediction of Odor and Flavor Intensity Ratings in the Elderly and Young.” Chem Senses 28, E1-E69, 2003.
71. Wille, C., Ostermann, M., Kettenmann, B., Kobal, G., “Influence of Flavors on the Perception of Video Clips.” Chem Senses 28, E1-E69, 2003.
72. Ackermen, D., “A Natural History of the Senses.” New York: Random House, Pg: 5. 1990.
73. Shoji, K., Taguchi, S., “The Effect of Fragrance on Weight Estimation.” Product Development Division, Shiseido Co., Ltd, 2-2-1 hayabuchi, Tsuzki-ku, Yokohama-shi 224-8558, Japan. The 36th Annual Meeting of the Japanese Association for the Study of Taste and Smell (JASTS XXXVI), Held in Kagoshima, Japan, Oct. 1-3, 2002. Chem Senses 28: J1-J35, 2003. Oxford University Press, 2003.
74. Wang J., Eslinger P. J., Smith M. B., Yang Q. X., “FMRI Study of Human Olfaction and Normal Aging at High Speed”, ACHemS-25th Annual Meeting: 2003, Abst. 223, p. 56 Kobal G., Wille C., Mueller C., Kettenmann B., “Olfactory Evoked Potentials During Monorhinal Versus Birhinal Stimulation with Linalool”, AChemS-25th Annual Meeting: 2003, Abst. 374, p. 93.
75. Redd W., Manne S., Peters B., Jacobson P., Schmidt H., “Fragrance Administration to Reduce Anxiety during MR Imaging”, JMRI 1994; 4:623-626.
76. Wrzesniewski A., McCauley C., Rozin P., “Odor and Affect: Individual Differences in the Impact of Odor on Liking for Places, Things and People”, Chem. Senses 24: 713-721, 1991.
77. Geisler T., Hubener F., Laska M., “Age-Related Changes in Olfactory Performance: Sensory, Cognition and Respiratory Factors”, AChemS-25th Annual Meeting: 2003, Abst. 138, p. 35.
78. Espineli E., Calhoun-Haney R. M., Zizak V., Pirogovsky E., Bailie J., Espineli K., Murphy C., “Taste and Odor Thresholds in Healthy Middle-aged and Elderly Participants Over an Eight Year Time Span”, AChemS-25th Annual Meeting: 2003, Abst. 142, p. 36.
79. Ohsawa I., Hagihara Y., Yoshida M., Ishikawa S., Gotoh M., “A Clinical Report on Taste Lesions in Human Patients”, JASTS XXIV: Abst. 50, p. 198.
80. Smith D., Standing L., De Man A., “Verbal Memory Elicited by Ambient Odor”, Perceptual and Motor Skills, 1992, 74, 339-343.
81. Zucco, G., & Tressoldi, P. (September 1987). Hemispheric differences in odor recognition. Paper presented at the second conference of the European Society for Cognitive Psychology, Madrid, Spain.
82. Ludvigson H., Rottman T., “Effects of Ambient Odors of Lavender and Cloves on Cognition, Memory, Affect and Mood”, Chemical Senses Vol. 14 no. 4 pp. 525-536, 1989.
83. Schab F., “Odors and the Remembrance of Things Past”, Journal of Experimental Psychology. Learning, Memory, and Cognition 1990, Vol. 16, No. 4, 648-655.
84. Rotten, J. (1983). “Affective and Cognitive Consequences of Malodorous Pollution”. Basic and Applied Social Psychology, 4, 174-191.
85. Forgas J., Vargas P. (2000) “Effects of Mood on Social Judgment and Reasoning”. In Lewis M., Haviland-Jones J. (Eds.), Handbook of Emotions, 2nd Edition (pp. 350-365). New York: The Guilford Press.
86. Clark, M. S., & Isen, A. M. (1982). Towards understanding the relationship between feeling states and social behavior. In A. H. Hastorf & A. M. Isen (Eds.), Cognitive social psychology (pp. 73-108). New York: Elsevier/North-Holland.

What is claimed is:

1. A method of modifying perception of chronological age of a female individual, comprising the step of:
administering to a male individual for inhalation an effective amount of a composition comprising a hedonically negative odorant, wherein the male individual perceives the age of the female individual to be about 3-20% less than actual chronological age of the female individual.

2. The method of claim 1, wherein the composition comprises a grapefruit odorant as a dominant odor.

3. The method of claim 1, wherein the composition comprises a pink grapefruit odorant as a primary odor.

4. The method of claim 1, wherein the composition comprises a mixture of pink grapefruit, vanilla, and baby powder odorants.

5. The method of claim 1, wherein the composition comprises a suprathreshold but non-irritant concentration of the odorant.

6. An article of manufacture, comprising a container of a composition consisting essentially of a suprathreshold and non-irritant concentration of a grapefruit odorant such that, when inhaled by an individual, the composition is effective to alter the individual's perception of chronological age of another individual.

7. An article of manufacture, comprising a container of a composition comprising a mixture of a grapefruit odorant, a vanilla odorant, and a baby powder odorant in a concentration effective such that, when inhaled by a male subject, the male subject's perception of age of a female individual is altered.

8. The article of claim 7, wherein the composition is effective to alter the male subject's perception of the age of the female individual such that said age is perceived to be about 4-10 years less than the actual age of the female individual.

9. An article of manufacture, comprising: packaging material and, contained within the packaging material, a composition comprising a suprathreshold and non-irritant concentration of a hedonically negative odorant, wherein the packaging material comprises a label that indicates that the composition can be used for altering perception of chronological age of an female individual upon inhalation by a male individual.

10. The article of claim 9, wherein the composition comprises a grapefruit odorant as a primary odor.

11. An article of manufacture, comprising, packaged together:
a container of a composition comprising a grapefruit odorant in a concentration effective upon inhalation by a male individual, to alter the male individual's perception of age of a female individual; and
instructions for use of the composition for altering perception of chronological age of a female individual in a method comprising administering an effective amount of the composition to a male individual for inhaling.

12. The article of claim 11, wherein the composition consists essentially of a grapefruit odorant.

13. A composition consisting essentially of of a grapefruit odorant in a concentration effective to alter a male individual's perception of chronological age of a female individual upon inhalation by the male individual whereby the female individual's chronological age is perceived by the male individual as being less than the actual chronological age.

14. The composition of claim 13, wherein the composition is effective to alter the male individual's perception of chronological age of the female individual such that the chronological age is perceived to be about 4-10 years less than the actual chronological age.

15. The composition of claim 13, wherein the composition comprises a suprathreshold and non-irritant concentration of the grapefruit odorant.

16. A composition comprising a mixture of a grapefruit odorant, a vanilla odorant, and a baby powder odorant in concentrations effective to alter a male individual's perception of age of a female individual upon inhalation of the composition such that the female individual's age is perceived by the male individual to be at least about 7-10 years less than the actual chronological age.

17. A method of screening an odorant or a mixture of odorants for effectively altering perception of chronological age, comprising the steps of:
administering a composition consisting essentially of said odorant or mixture of odorants to a male individual for inhalation;
having the male individual estimate the chronological age of a female subject;
comparing the estimate of the chronological age to actual chronological age of the female subject to provide a difference value;
comparing the difference value to a control value to determine the statistical significance of the difference value; and
eliminating the odorant or odorant mixture as being ineffective for altering perception of age if not statistically significant.

18. The method of claim 17, wherein the control value is derived by having the male estimate the chronological age of the female subject without the composition administering step, and comparing the chronological age estimate with the actual chronological age of the female subject to provide the control value.

19. The method of claim 18, further comprising, prior to the composition administering step, the steps of having the male individual estimate the chronological age of the female subject, and comparing the chronological age estimate with the actual chronological age of the female subject to provide a control value.

20. The method of claim 19, further comprising the step of having the maleindividual identify the composition as hedonically positive or hedonically negative.
